SEQUENCE LISTING (65 sequences)

SEQ ID NO: 1 - protein, 18 aa, Trypanosoma cruzi
MLSKNVDYFDGLRTFPRC

SEQ ID NO: 2 - protein, 16 aa, Trypanosoma cruzi
AKPPAESPFKSVFGAC

SEQ ID NO: 3 - protein, 18 aa, Trypanosoma cruzi
DAVKLEEFRFFIYERPSC

SEQ ID NO: 4 - protein, 22 aa, Trypanosoma cruzi
EESAASRPLYSFEAESLASHRC

SEQ ID NO: 5 - protein, 18 aa, Trypanosoma cruzi
TEDGDFGWTVDVPIFLEC

SEQ ID NO: 6 - protein, 21 aa, Trypanosoma cruzi
KRCRDDTTPEYILSLGPQSAC

SEQ ID NO: 7 - protein, 16 aa, Trypanosoma cruzi
SSTEIETFFLEGMSEC

SEQ ID NO: 8 - protein, 20 aa, Trypanosoma cruzi
AVSQLGEVENIKNKYSLHIC

SEQ ID NO: 9 - protein, 20 aa, Trypanosoma cruzi
RITNFILEGRHAHEKDGQGC

SEQ ID NO: 10 - protein, 16 aa, Trypanosoma cruzi
TTYPPEDVYFPELRFC

SEQ ID NO: 11 - protein, 16 aa, Trypanosoma cruzi
LDAHQLEINRHLFESC

SEQ ID NO: 12 - protein, 20 aa, Trypanosoma cruzi
VADDGRVTPFYYDFEPLLAC

SEQ ID NO: 13 - protein, 17 aa, Trypanosoma cruzi
PPFKETQFSYFPSSDSC

SEQ ID NO: 14 - protein, 16 aa, Trypanosoma cruzi
ENDGYTQEHGIFQFGC

SEQ ID NO: 15 - protein, 16 aa, Trypanosoma cruzi
GDLIRRTLEEFSGDEC

SEQ ID NO: 16 - protein, 17 aa, Trypanosoma cruzi
KRIVSNMVPEIVDWVNC

SEQ ID NO: 17 - protein, 21 aa, Trypanosoma cruzi
GVVVFDLLRNEFTNEIFIDEC

SEQ ID NO: 18 - skipped

SEQ ID NO: 19 - protein, 16 aa, Trypanosoma cruzi
DKAYEWFPDSIQFLTC

SEQ ID NO: 20 - protein, 18 aa, Trypanosoma cruzi
NDVLTSRGETYDYQYSWC

SEQ ID NO: 21 - protein, 16 aa, Trypanosoma cruzi
RFRTAHEDSFYEFIFC

SEQ ID NO: 22 - protein, 16 aa, Trypanosoma cruzi
GYPEKPRDLFHEYLFC

SEQ ID NO: 23 - protein, 21 aa, Trypanosoma cruzi
RNMREESQTVDEFLFKRVLLC

SEQ ID NO: 24 - protein, 18 aa, Trypanosoma cruzi
SHTDLHFHAFQENYEDWC

SEQ ID NO: 25 - protein, 16 aa, Trypanosoma cruzi
AEAQHAMFDDDFLLFC

SEQ ID NO: 26 - protein, 19 aa, Trypanosoma cruzi
RNSASFLSGGEFNDWSNHC

SEQ ID NO: 27 - protein, 22 aa, Trypanosoma cruzi
VQDGDTKNTPGAFFFAELKGGC

SEQ ID NO: 28 - protein, 16 aa, Trypanosoma cruzi
SALQPEHDELFFYRFC

SEQ ID NO: 29 - protein, 16 aa, Trypanosoma cruzi
DNFSGDATAGDRKVNC

SEQ ID NO: 30 - protein, 17 aa, Trypanosoma cruzi
DIATLRDGGYDFFIENC

SEQ ID NO: 31 - protein, 18 aa, Trypanosoma cruzi
AELLCGNAGNRKVNSLAC

SEQ ID NO: 32 - protein, 22 aa, Trypanosoma cruzi
DVAAKLQHPEFLFSDEDSVLLC

SEQ ID NO: 33 - protein, 16 aa, Trypanosoma cruzi
KLDVAFDFEVDAFLFC

SEQ ID NO: 34 - protein, 16 aa, Trypanosoma cruzi
MLREESPIIQMVWDAC

SEQ ID NO: 35 - protein, 16 aa, Trypanosoma cruzi
RTNESIFLENSEKWFC

SEQ ID NO: 36 - protein, 17 aa, Trypanosoma cruzi
NINAGREDILLFPLSRC

SEQ ID NO: 37 - protein, 16 aa, Trypanosoma cruzi
PTSTDDEFQLIQLIQC

SEQ ID NO: 38 - protein, 16 aa, Trypanosoma cruzi
PAPFGQAAAGDKPSPC

SEQ ID NO: 39 - protein, 20 aa, Trypanosoma cruzi
DKPSPFGQAAAGDKPPPFGC

SEQ ID NO: 40 - protein, 20 aa, Trypanosoma cruzi
SAHSTPSTPVDSSAHSTPSC

SEQ ID NO: 41 - protein, 16 aa, Trypanosoma cruzi
AEEEEDDDMGFGLFDC

SEQ ID NO: 42 - protein, 17 aa, Trypanosoma cruzi
GTDKKTAAGEAPSPSGC

SEQ ID NO: 43 - protein, 16 aa, Trypanosoma cruzi
AKAAAAPAKAAAAPAC

SEQ ID NO: 44 - protein, 16 aa, Trypanosoma cruzi
EEEDDDDDFGMGALFC

SEQ ID NO: 45 - protein, 16 aa, Trypanosoma cruzi
ETEKQKAAEATKVAEC

SEQ ID NO: 46 - protein, 16 aa, Trypanosoma cruzi
QEDVGPRHVDPDHFRC

SEQ ID NO: 47 - protein, 16 aa, Trypanosoma cruzi
PEGVPLRELPLDDDSC

SEQ ID NO: 48 - protein, 16 aa, Trypanosoma cruzi
DFQETRPIPVWDLVYC

SEQ ID NO: 49 - protein, 16 aa, Trypanosoma cruzi
KGAPLYDDVFVPSFDC

SEQ ID NO: 50 - protein, 16 aa, Trypanosoma cruzi
VEPVCDDTQEYYFRLC

SEQ ID NO: 51 - protein, 16 aa, Trypanosoma cruzi
EIYLETIRNATNETIC

SEQ ID NO: 52 - protein, 16 aa, Trypanosoma cruzi
TALRVSPYSIFLQELC

SEQ ID NO: 53 - protein, 16 aa, Trypanosoma cruzi
AAKPPAESPFRSVFGC

SEQ ID NO: 54 - protein, 16 aa, Trypanosoma cruzi
KPHAESPFKSVFGAPC

SEQ ID NO: 55 - protein, 16 aa, Trypanosoma cruzi
HSPRGSCKALLPEDAC

SEQ ID NO: 56 - protein, 16 aa, Trypanosoma cruzi
LRCVDSALEEAHLSIC

SEQ ID NO: 57 - protein, 16 aa, Trypanosoma cruzi
QFVPPWLQGYAFLPEC

SEQ ID NO: 58 - protein, 16 aa, Trypanosoma cruzi
LRESDFVVNILPGTEC

SEQ ID NO: 59 - protein, 16 aa, Trypanosoma cruzi
EALTKDDTGNFWYSFC

SEQ ID NO: 60 - protein, 16 aa, Trypanosoma cruzi
VPKLFDLTYIPETSEC

SEQ ID NO: 61 - protein, 16 aa, Trypanosoma cruzi
AETAEPKEFFFDVDRC

SEQ ID NO: 62 - protein, 16 aa, Trypanosoma cruzi
GHLDDRVQIPDLFTLC

SEQ ID NO: 63 - protein, 16 aa, Trypanosoma cruzi
GDARDWDILLAVGEVC

SEQ ID NO: 64 - protein, 16 aa, Trypanosoma cruzi
HKNDEGVTVSLVESLC

SEQ ID NO: 65 - protein, 16 aa, Trypanosoma cruzi
VTREDEEYIVFSPQHC

(19) United States
(12) Patent Application Publication
(10) Pub. No.: US 2025/0138007 A1
(43) Pub. Date: May 1, 2025

(54) ANTIGENIC PEPTIDES AND METHODS OF USE FOR DIAGNOSIS OF CHAGAS DISEASE

(71) Applicant: THE ADMINISTRATORS OF THE TULANE EDUCATIONAL FUND, New Orleans, LA (US)

(72) Inventors: Eric Dumonteil, New Orleans, LA (US); Claudia Herrera, New Orleans, LA (US)

(21) Appl. No.: 18/718,745

(22) PCT Filed: Dec. 16, 2022
(86) PCT No.: PCT/US22/81738

§ 371 Date: Jun. 11, 2024

Related U.S. Application Data
(60) Provisional application No. 63/290,972, filed on Dec. 17, 2021.

(51) Int. Cl. G01N 33/569; C07K 14/44; G01N 33/68

(52) U.S. Cl. CPC G01N 33/56905; C07K 14/44; G01N 33/6854; G01N 2333/44; G01N 2469/20

(57) ABSTRACT

The present disclosure provides novel antigenic peptides and peptide mixtures, and methods of use for the detection of T. cruzi antibodies in tissue samples and the diagnosis of Chagas disease in a subject.

### RELATED APPLICATIONS

This application claims the benefit of U.S. Provisional Application Ser. No. 63/290,972 filed Dec. 17, 2021, the entire disclosure of which is incorporated herein by reference.

### GOVERNMENT INTEREST

This invention was made, in part, with support provided by the United States government under Grant No. R01HD094955 awarded by the National Institutes of Health, Eunice Kennedy Shriver National Institute of Child Health and Human Development. The United States government has certain rights in this invention.

### REFERENCE TO SEQUENCE LISTING SUBMITTED ELECTRONICALLY

A sequence listing electronically submitted with the present application as an XML file named Antigenic_Peptides_for_Chagas_Seq.xml, created on Dec. 16, 2022 and having a size of 56000 bytes, is incorporated herein by reference in its entirety

### TECHNICAL FIELD

The disclosure relates to antigens for use in serological tests for the identification of Trypanosoma cruzi in tissue samples. The present invention further relates to methods of identification of Chagas disease in subjects.

### BACKGROUND

Chagas Disease is a neglected tropical disease that causes 10-15 thousand deaths and hundreds of millions of dollars in economic losses globally each year. The etiological agent Trypanosoma cruzi (T. cruzi) comprises great diversity. Current nomenclature describes seven distinct genetic lineages, or Discrete Typing Units (DTUs), namely TcI-TcVI and TcBat, with a growing body of evidence of both intra-lineage diversity and geographic structuring of diversity. This may contribute to observed poor serological diagnostic performance, which is prominent in Central and North America, with current diagnostics developed and evaluated nearly exclusively in South America using both parasite strains and human samples from a limited geographic range. Early and accurate diagnosis is important to improve outcomes and to reduce transmission. Thus, we identified conserved coding regions of T. cruzi through genomic analyses, identified antigenic linear sequences using a peptide microarray, and evaluated diagnostic performance of new peptidic antigens for use in an improved serological diagnostic.

As drug therapy is not efficacious in the chronic phase of disease, it is important to have rapid and accurate diagnostics, yet no single point-of-care test developed thus far has proven sufficient for standalone use. Poor diagnostic performance also creates a higher potential risk for both congenital transmission and transfusion/transplant transmission of Chagas Disease. It would be useful to have diagnostic antigens and methods with improved serological diagnostic performance across geographic areas impacted by Chagas Disease. The present invention fills these and other needs.

### SUMMARY

In accordance with the purposes and benefits described herein, novel compositions are described, comprising a peptide or a peptide mixture having antigenic properties with respect to Trypanosoma cruzi (T. cruzi). The composition comprises at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65.

In alternative embodiments, the composition comprises at least one analog, homolog, or antigenic fragment of one or more of SEQ ID NO:1-SEQ ID NO:65, or a peptide or peptide mixture having at least 70% sequence identity to one or more of SEQ ID NO:1-SEQ ID NO:65, or a combination thereof.

In embodiments, the compositions comprise a peptide mixture consisting of SEQ ID NO:2, 6, 10, 26, 51, and 55, or at least one analog, homolog, or antigenic fragment of one or more of the group of sequences consisting of SEQ ID NO:2, 6, 10, 26, 51, and 55, or a peptide or peptide mixture having at least 70% sequence identity to one or more of the group of sequences consisting of SEQ ID NO:2, 6, 10, 26, 51, and 55, or a combination thereof.

In alternative embodiments, the compositions comprise a peptide mixture consisting of SEQ ID NO:2, 7, 9, 10, 26, 29, 34, and 61, or at least one analog, homolog, or antigenic fragment of one or more of the group of sequences consisting of SEQ ID NO:2, 7, 9, 10, 26, 29, 34, and 61, or a peptide or peptide mixture having at least 70% sequence identity to one or more of the group of sequences consisting of SEQ ID NO:2, 7, 9, 10, 26, 29, 34, and 61, or a combination thereof.

In other alternative embodiments, the compositions comprise a peptide mixture consisting of SEQ ID NO:SEQ ID NO:2, 7, 9, 10, 26, 29, 34, 54, and 58, or at least one analog, homolog, or antigenic fragment of one or more of the group of sequences consisting of SEQ ID NO:2, 7, 9, 10, 26, 29, 34, 54, and 58, or a peptide or peptide mixture having at least 70% sequence identity to one or more of the group of sequences consisting of SEQ ID NO:2, 7, 9, 10, 26, 29, 34, 54, and 58, or a combination thereof.

In still other alternative embodiments, the compositions comprise a peptide mixture consisting of SEQ ID NO:2, 7, 9, 10, 12, 20, 26, 29, 34, 38, 52, 53, 54, and 58, or at least one analog, homolog, or antigenic fragment of one or more of the group of sequences consisting of SEQ ID NO:2, 7, 9, 10, 12, 20, 26, 29, 34, 38, 52, 53, 54, and 58, or a peptide or peptide mixture having at least 70% sequence identity to one or more of the group of sequences consisting of SEQ ID NO:2, 7, 9, 10, 12, 20, 26, 29, 34, 38, 52, 53, 54, and 58, or a combination thereof.

In still yet other alternative embodiments, the compositions comprise a peptide mixture consisting of SEQ ID NO:6, 7, 9, 10, 12, 15, 20, 26, 29, 31, 34, 38, 52, 53, 54, 55, 58, 62, and 65, or at least one analog, homolog, or antigenic fragment of one or more of the group of sequences consisting of SEQ ID NO:6, 7, 9, 10, 12, 15, 20, 26, 29, 31, 34, 38, 52, 53, 54, 55, 58, 62, and 65, or a peptide or peptide mixture having at least 70% sequence identity to one or more of the group of sequences consisting of SEQ ID NO:6, 7, 9, 10, 12, 15, 20, 26, 29, 31, 34, 38, 52, 53, 54, 55, 58, 62, and 65, or a combination thereof.

In still yet other alternative embodiments, the compositions comprise a peptide mixture consisting of SEQ ID NO:6, 7, 9, 10, 12, 15, 20, 26, 29, 31, 34, 39, 43, 44, 45, 52, 53, 54, 55, 58, 62, and 65, or at least one analog, homolog, or antigenic fragment of one or more of the group of sequences consisting of SEQ ID NO:6, 7, 9, 10, 12, 15, 20, 26, 29, 31, 34, 39, 43, 44, 45, 52, 53, 54, 55, 58, 62, and 65, or a peptide or peptide mixture having at least 70% sequence identity to one or more of the group of sequences consisting of SEQ ID NO:6, 7, 9, 10, 12, 15, 20, 26, 29, 31, 34, 39, 43, 44, 45, 52, 53, 54, 55, 58, 62, and 65, or a combination thereof.

In still yet other alternative embodiments, the compositions comprise a peptide mixture consisting of SEQ ID NO:6, 7, 9, 10, 12, 20, 26, 29, 31, 34, 39, 43, 45, 52, 53, 54, 55, 58, 62, and 65, or at least one analog, homolog, or antigenic fragment of one or more of the group of sequences consisting of SEQ ID NO:6, 7, 9, 10, 12, 20, 26, 29, 31, 34, 39, 43, 45, 52, 53, 54, 55, 58, 62, and 65, or a peptide or peptide mixture having at least 70% sequence identity to one or more of the group of sequences consisting of SEQ ID NO:6, 7, 9, 10, 12, 20, 26, 29, 31, 34, 39, 43, 45, 52, 53, 54, 55, 58, 62, and 65, or a combination thereof.

In another aspect, the present disclosure provides a method for detecting a T. cruzi antibody in a biological sample, comprising steps of obtaining a biological sample from a subject, contacting the biological sample with the composition according to the disclosure, and detecting and/or quantifying the T. cruzi antibody bound to the composition. In embodiments, the biological sample may be selected from the group consisting of tissue, blood, saliva, urine, and serum. Any suitable immunoassay may be availed to detect and/or quantify the T. cruzi antibody.

In yet another aspect, the present disclosure provides method for diagnosing Chagas disease and/or a T. cruzi infection, comprising the above method. Additional confirmatory diagnostic analyses may be employed to confirm the initial diagnosis of Chagas disease and/or T. cruzi infection. In embodiments, the subject may be selected from a group of subjects having or at risk of having one of an acute phase of Chagas disease, a chronic phase of Chagas disease, an indeterminate phase of Chagas disease, or a congenital T. cruzi infection. The subject may have previously tested serologically negative for T. cruzi.

In still yet another aspect, the present disclosure describes an apparatus for determination and/or quantification of one or more T. cruzi antibodies. The apparatus comprises a suitable substrate and the composition according to the disclosure, wherein the peptide or peptide mixture is immobilized on the suitable substrate to provide a microarray. In embodiments, the peptide or peptide mixture may be conjugated to a suitable carrier protein for the immobilization. In embodiments, the suitable substrate is provided by one or more wells of a microtiter plate or an ELISA microplate, or a rapid immunochromatographic test.

In still yet another aspect, a kit for determination and/or quantification of one or more T. cruzi antibodies is provided. The kit may comprise the apparatus described above, and optionally, packaging, one or more suitable preservatives for the peptide or peptide mixture, and instructional materials for the use of the apparatus.

In still yet another aspect, the present disclosure provides a method for detecting T. cruzi contamination, comprising obtaining a biological sample and contacting the biological sample with the composition according to the disclosure. One or more T. cruzi antibodies bound to the composition may then be detected by any suitable immunoassay.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows an example view of peptide microarray results. Figure shows results from one slide with Discordant Positive pool probe. Enlarged view demonstrates evident differences in reactivity across peptides.

FIG. 2 shows heatmaps indicating the antibody binding to a subset of 4,700 T. cruzi peptides from the high-density microarray. These peptides represented those for which binding was the most variable across the three samples tested, which included negative (NEG) and positive controls (POS), as well as positive but serologically discordant samples (DPos). Antibody binding profile to the peptides varied for each sample, and peptides were grouped into 4 subsets according to their binding profile (groups 1-4). Group 1 (106 peptides) had a high binding with the negative and positive controls, and are thus not specific for T. cruzi infection. Group 2 (2256 peptides) presented binding from only the positive control, but no binding from the negative control or the discordant positive samples. Group 4 (235 peptides) presented binding from the discordant positive samples only, and no binding from the positive control samples. Finally, group 3 (2176 peptides) showed specific binding from both the positive control and discordant positive samples. Enlarged views of the heatmaps are shown for a subset of peptides for groups 2, 3 and 4. Binding intensity is color-coded according to the indicated scale in the graph, with green corresponding to no/low binding, and red corresponding to high binding.

FIGS. 3A-3F show reactivity profiles of some antigens from current commercial tests on the high-density microarray. Heat maps show relative intensity for binding profiles of all three pools across all 15-mers comprising each peptide. Y-axis indicates individual 15-mers and intensity of binding to each peptide is color-coded according to the indicated scale, with blue representing low binding and yellow high binding. Antigen names are indicated on each panel. These antigens do not produce a strong response to the positive yet discordant pool (DP), despite a robust response to the unequivocally positive pool (POS). While a few peptides showed non-specific background binding in the negative pool (NEG), nearly all of the peptides across these six proteins demonstrated no to very low signal in response to the NEG control.

FIGS. 4A-4D show response of samples to antigen variants covering different parasite strains and DTUs. Antibodies from the positive control (POS) are able to bind to these antigens irrespective of the strain or DTU they are derived from. Strikingly, antibodies from discordant but positive samples (DP) failed to bind to all antigen sequence variants tested for B13, antigen 30 and JL8, and only showed limited binding to SAPA, but again this binding profile was very similar across strains and DTUs and always much lower compared to that obtained for the positive sample control. No binding was observed for the negative control (NEG).

FIG. 5 shows heatmaps summarizing the reactivity (red) and non-reactivity (green) of samples against various peptides. The top part corresponds to 11 peptides derived from well characterized antigens, some of which are used in current commercial tests. The bottom part corresponds to peptides identified as antigenic in our microarray. Individual plasma samples tested included negative and positive controls, as well as discordant samples.

FIG. 6 shows heatmaps summarizing the reactivity (red) and non-reactivity (green) of the same set of plasma samples of FIG. 5 against various peptide combinations including mixtures ranging from 6 to 22 peptides. Performance was also compared to the FDA approved Wiener ELISA.

FIG. 7 shows improved sensitivity and specificity of a particular peptide mixture (14), observed in samples from all countries tested (Argentina, Honduras and Mexico).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

In one embodiment, the present invention comprises one or more antigenic peptides selected or designed to have antigenic properties with respect to T. cruzi. In certain embodiments, the present invention comprises a composition comprising at least one such peptide, wherein the composition is an analog, homolog, or fragment of such peptide or has at least 70% sequence identity to the sequence of at least one such peptide. Certain embodiments include compositions having antigenic properties with respect to T. cruzi.

In another embodiment, the present invention comprises a method of detecting one or more T. cruzi antibodies in a tissue sample, including a blood, saliva, urine, and serum sample, comprising the steps of: obtaining a sample sufficient for serological analysis from a subject; contacting the sample with one or more such peptides of or compositions; and performing an immunoassay to determine the presence or concentration of one or more T. cruzi antibodies.

In another embodiment, the present invention comprises a method of diagnosing T. cruzi infection in a subject comprising the steps of: obtaining a sample sufficient for serological analysis from a subject; contacting the sample with one or more such peptides or compositions; performing an immunoassay to determine the presence or concentration of one or more T. cruzi antibodies; and performing additional diagnostic analysis based on the presence or concentration of one or more T. cruzi antibodies. The present invention includes embodiments in which such method is suitable for a subject is in the acute, indeterminate, or chronic phase of Chagas disease infection or with congenital infection with T. cruzi. Further, the present invention includes embodiments in which such method is suitable for a subject for whom one or more previous samples were determined to be serologically negative for the presence of T. cruzi.

In another embodiment, the present invention comprises an article comprising one or more of such peptides or compositions. Such embodiment comprises articles in which such peptide or peptides are immobilized on a substrate surface to form a microarray, are chemically conjugated with a carrier protein (including bovine serum albumin) to be immobilized in the wells of ELISA microplates, or are analyzed by rapid diagnostic test.

In another embodiment, the present invention comprises a kit for diagnosis of T. cruzi infection comprising: such article; materials for preservation and packaging of such article; and information for conducting and interpreting the results of an assay conducted with such article.

In another embodiment, the present invention comprises a method for detecting contamination of a tissue sample, including a blood, saliva, urine, and serum sample, with T. cruzi, the method comprising: selecting a sample; contacting the sample with one or more such peptides, compositions, or articles; and determining the presence or concentration of one or more T. cruzi antibodies of the sample to determine whether the blood sample is contaminated with T. cruzi.

In another embodiment, the present invention comprises a method of designing a diagnostic test, including a serological test, comprising one or more peptides selected from such peptides or compositions. In certain embodiments, such method comprises a diagnostic test that allows for diagnosis of Chagas disease infection resulting from one or more areas within the geographic range impacted by Chagas disease or from one or more distinct genetic lineages or Discrete Typing Units of T. cruzi. In certain embodiments, the present invention includes genomic information from strains representing all DTUs to identify new antigens that are highly conserved. A unique biorepository of very well-characterized samples, previously evaluated on several serological and molecular tests, provided innovative insight into discordant samples and a rigorous gold standard for sensitivity to evaluate newly defined candidate antigens.

The presently-disclosed subject matter is further illustrated by the following specific but non-limiting examples. The following examples may include compilations of data that are representative of data gathered at various times during the course of development and experimentation related to the present invention.

### EXAMPLES

### Materials and Methods

Identification of Highly Conserved Trypanosoma cruzi Epitopes Towards Improved Diagnosis for Chagas Disease

### Peptide Selection

Fourteen T. cruzi genomes representing different geographies and DTUs (Table 1) were aligned using progressiveMAUVE to identify homologous regions across genomes, or locally colinear blocks (LCBs), using either current assemblies or newly assembled sequence reads. Current annotations were used to identify coding region annotations across these regions and those annotated regions with good sequencing coverage across 2-14 genomes were extracted from the alignment. Extractions with >80% nucleotide pairwise identity across all genomes represented were selected for further analysis.

TABLE 1
Subset of T. cruzi genomes aligned to identify conserved regions

| Genome | DTU | Country of | Host of Origin | Accession No. |
|---|---|---|---|---|
| WB1 | TcI | United States | Triatomine | unpublished |
| Corpus_Christi | TcI | United States | Human | SRX1054555 |
| H1_Yuc | TcI | Mexico | Human | SRX1851500 |
| Bug2148 | TcI | Brazil | Human | NMZN00000000 |
| Dm28c | TcI | Colombia | Opossum | GCA_003177105.1 |
| Esmeraldo | TcII | Brazil | Human | SRX022423, SRX022425, |
| | | | | SRX022426, SRX022427, |
| | | | | SRX022428, SRX022429, |
| | | | | SRX2015243, SRX2015244 |
| Y | TcII | Brazil | Human | SRR1796718, SRR1797819 |
| 231 | TcIII | Brazil | Human | OGCJ00000000 |
| Canill | TcIV | Brazil | Human | SRR1996498, SRR1996501 |
| SC43 | TcV | Bolivia | Triatomine | GCA_015455285.1 |
| 9280d2 | TcV | Bolivia | Human | SRR1996492, SRR1996493, |
| | | | | SRR1996496, SRR1996497, |
| | | | | SRR1996502 |
| TCC | TcVI | Chile | Human | GCA_003177095.1 |
| Tulacl2 | TcVI | Chile | Human | SRX268890, SRX268891, |
| | | | | SRX268892, SRX268893, |
| | | | | SRX268894, SRX268895, |
| | | | | SRX268896 |
| H1 | TcVI | Panama | Human | SRR3676277 |

Nucleotide sequences were next translated in an appropriate reading frame and resulting protein sequences were aligned via MUSCLE. If a protein sequence from any genome in the alignment contained a stop codon prior to the full length of the sequence, it was inferred that such changes were more likely attributed to sequencing errors rather than reflecting a true biological change. Such sequences were, thus, edited using the alternative translation frame that corrected the remaining length of the sequence when possible or otherwise removed from the alignment. Protein alignments with !90% pairwise identity were kept in consideration, while those below this threshold were discarded, and the consensus peptide sequence for each alignment was extracted. All selected protein consensus sequences were then compared to databases created from other parasite genomes (Table 2), including Leishmania spp., Trypanosoma rangeli, Trypanosoma brucei, Plasmodium vivax, and Toxoplasma gondii, using tBLASTn (AA inquiry of a DNA database), to ensure that these sequences would be specific for T. cruzi and not cross-react with other pathogens. These species were selected for overlap of disease geography and/or relatedness to T. cruzi. Sequences were also compared to a Homo sapiens genome (Table 2) similarly via BLAST to ensure there would be no signal from host background.

TABLE 2
Genomes Used for Specificity BLAST search

| Species | Accession Number(s) |
|---|---|
| Trypanosoma rangeli | GCA_003719475.1 |
| Trypanosoma brucei brucei, | GCA_000002445.1, GCA_000210295.1 |
| Toxoplasma gondii | GCA_000006565.2 |
| Plasmodium vivax | GCA_000320645.2, GCA_000002415.2 |
| Leichmania infantum, Leichmania | GCA_000002875.2, GCA_000227135.2, |
| donovani, Leichmania brazillensis, | GCA_000002845.2, GCA_902369315.1, |
| Leichmania guyanensis, Leichmania | GCA_902369305.1, GCA_000124665.4, |
| adleri, Leichmania mexicana, | GCA_000755165.1, GCA_000002725.2, |
| Leichmania panamensis, Leichmania | GCA_014466935.1, GCA_009731335.1, |
| major, Leichmania chagasi, Leichmania | GCA_005317125.1, GCA_003664395.1, |
| terentolae, Leichmania amazonensis, | GCA_001403675.1, GCA_000981925.2 |
| Leichmania lainsoni, Leichmania | |
| peruviana | |
| Homo sapien | GCA_000001405.28 |

Pairwise identity of BLAST matches, along with a “grade” of the BLAST hit assigned by Geneious based on the pairwise identity, E value, and length of the hit, were used to filter sequences. Sequences with a BLAST return in any of the databases created from other parasite or human genomes of >90% pairwise identity were discarded, along with those hits that had a grade of >90% plus a pairwise identity of >85%. A few markers with a pairwise identity to T. rangeli >90% were not discarded due to their low (<50%) grade, as only a small portion of the peptide sequence shared this degree of identity. All remaining unique consensus protein sequences (n=1573) were considered conserved candidate antigens and used to design an overlapping peptide microarray of 15mers with an overlap of 13aas.

### Human Sample Probes

Human serum samples derived from a biorepository obtained through previous research. These samples were previously characterized using several diagnostic approaches, including both direct and indirect detection methods.

Samples were previously tested with Chagas STAT PAK®, Trypanosoma Detect™, Chagatest ELISA recombinante v. 3.0, and Hemagen® Chagas' Kit as well as by quantitative PCR and two qualitative PCR protocols, one targeting nuclear satellite DNA (primers TcZ1-TcZ2), and one targeting minicircle DNA (primers 121-122).

Three pools were created for microarray probing (Table 3): A pool of unequivocally positive samples (POS), a pool of unequivocally negative samples (NEG), and a pool of samples positive by PCR and serologically discordant in that each was only positive on one of the four serological tests used (DP).

TABLE 3
Three pools of human sera

| | | | Chagatest | | | | |
|---|---|---|---|---|---|---|---|
| | Chagas | | ELISA | Hemagen ® | | | |
| | STAT | Trypanosoma | recombinante | Chagas' | PCR | PCR | |
| Pool | PAK ® | Detect ™ | v3.0 | Kit | TCZ | 121-22 | qPCR |
| POS | 9/9 | 9/9 | 9/9 | 8/9 | 8/9 | 8/9 | 7/9 |
| NEG | 0/11 | 0/11 | 0/11 | 1/11 | 0/8 | 0/8 | ND |
| DP | 7/11 | 2/11 | 0/11 | 1/11 | 6/11 | 6/11 | 11/11 |

Data are presented as number of reactive samples/total number of samples tested in each pool.

The positive pool consisted of nine individual samples, and all other pools each consisted of eleven individual samples. For each pool, two microliters of serum per individual sample were combined and IgG antibody was purified using the Thermo Scientific™ Melon™ Gel IgG Spin Purification Kit, per kit instructions.

Concentration and purity of resulting IgG were measured using the A280 protein method on a Nanodrop2000 spectrophotometer (Table 4).

TABLE 4
Concentration and Purity of Human IgG Pools

| Pool | IgG Concentration (mg/mL) | OD 260/280 |
|---|---|---|
| POS | 1.001 | 0.52 |
| NEG | 1.421 | 0.50 |
| DP | 1.104 | 0.54 |

### Microarray

PepArray was used to design an overlapping peptide library of 15-mers, offset by 2 aa from those conserved, specific proteins selected above and a series of controls including previously reported T. cruzi antigens, neoproteins of similar biochemistry to T. cruzi. Approximately 500,000 unique, overlapping 15-mers were synthesized on microarray slides along with additional controls of multiple copies of the “SIRANETIYNTTLKY” herpes envelope glycoprotein sequence as well as overlapping 15-mers from Human Cytomegalovirus Strain AD169 large structural phosphoprotein and blank Cy3 marker.

In short, one copy of each unique 15-mer was synthesized on a functionalized slide with a D(bAla)D linker by Schafer-N(Denmark) in a 20×20 μm layout with 10 μm separation. Slides were deprotected in TFA EDT H20 for 3 h at room temperature and blocked overnight in 0.1% BSA, 0.1% Tween-20 in PBS. After blocking, slides were incubated for 1 h at RT with one of the three probes described above (100 μg/ml in 0.1% BSA, 0.1% Twee n-20 in PBS), washed 3×20 min with 0.1% BSA, 0.1% Tween-20 in PBS and incubated for 1 h at RT with Cy3-goat anti-Hu IgG (1 Vg/ml in 0.1% BSA, 0.1% Tween-20 in PBS). Slides were washed 3×20 min with 0.1% BSA in PBS, dried, and scanned using a laser scanner with 1 μm resolution. Assuming a normal distribution of signals from fields with no sequence, the mean blanks for each sector of the array slide were subtracted from the individual peptide signals in each respective sector, and z-scores were determined as the number of blank-standard deviations the peptide signal is above the blank-mean for that sector. These z-scores were used to assign p-values, or probability that the peptide signal is not significant.

### Microarray Analysis

The intern controls described above were evaluated and compared across slides and pool groups to ensure that data was valid and comparable. Response to positive T. cruzi controls of previously reported antigens was also compared across the three pools, as well as response to the three neoproteins included as negative peptide controls. As many of the previously reported antigens showed differential profiles across the three groups, we chose to focus on a subset of these for further analysis, including antigens in currently used diagnostics. Individual peptides were mapped back to full length proteins to create binding profiles across these proteins, as well as potential new candidate antigens, for the three different pools.

### Selection of Candidate Peptides

Peptides were first sorted by microarray signal values according to the largest difference in signal between the DP and NEG human pools. The hundred peptides that showed the largest signal difference between these two groups were submitted to a protein BLAST with parameters adjusted for short sequence input. Peptides with the lowest percent identities to any other species, including other Trypanosoma species, as well as the highest E-values (>2 except for one exception) were selected. Those with predicted good aqueous solubility were prioritized. Along with novel peptides identified through our genomic analyses, we also selected previously reported antigens, such as B13, to evaluate their performance in downstream ELISA testing with our unique biorepository of samples.

Evaluation of Novel Trypanosoma cruzi Peptidic Antigens in ELISA Assays

### Samples

Human samples utilized for this study again derived from the biorepository previously described herein. To validate the microarray data, we again used the three human pools described above. Once validation was performed, each of these 42 samples were tested individually against the candidate peptides.

### Peptides

Peptides were synthesized by Peptide 2.0 (Chantilly, VA) with an additional C-terminal cysteine residue and conjugated to maleimide-activated BSA (Thermo-Fisher) following manufacturer's protocols. Briefly, 0.5 mg of peptide was mixed with 0.5 mg of carrier protein and incubated at room temperature for 2 hours. The mixture was then applied to a Zeba desalting column (Thermo Fisher) to remove excess unbound peptide and final protein concentration was measured using Nanodrop ProteinA280.

### ELISA Assays

ELISA conditions were optimized by testing a series of dilutions with the protocol below. Samples were initially diluted 1:500 and 1:1,000 and the secondary antibody was diluted 1:5,000, 1:10,000, 1:20,000, 1:40,000, and 1:80,000, with all dilutions made into PBS with 0.05% Tween and 0.3% BSA. After testing these initial conditions, samples were diluted 1:500 and secondary antibody was diluted 1:80,000 for all further ELISA assays. Initially, each peptide was tested against the same pools described above in the ELISA format. After this validation of the microarray, the 42 samples comprising the three pools were tested individually. Conjugated peptides were appropriately diluted to 0.5 ng/ul in PBS, pH 7.4 and coated onto 96-well polystyrene plates overnight at 4° C. at a volume of 200 ul to coat 100 ng. Unbound peptide was removed by sequential washes with a PBST wash buffer and plates blocked for 1 h at room temperature with 200 ul PBS with 0.05% Tween and 1% BSA. After another series of sequential washes, plates were incubated with 100 ul of diluted human plasma samples (1:500) for 1 h at room temperature and again washed. A horseradish peroxidase-labeled goat anti-human IgG secondary antibody (1:80,000) was added to a volume of 100 ul and incubated for 1 h at room temperature. Finally, 200 ul/well of substrate solution (Phosphate Citrate Buffer+TMB+35% fresh hydrogen peroxide) was incubated in the dark at room temperature for 30 minutes. The reaction was stopped using 2M H2SO4 and plates were read at 450 nm.

### ELISA Analysis

OD readings were adjusted for each plate by subtracting the average blank read from all individual reading values. GraphPad Prism9 was used to construct Receiver Operating Characteristic (ROC) curves for each peptide and the optimum cutoff was defined as the mean OD reading of negative samples plus two standard deviations. ROC curves were also constructed for various combinations of peptides, including the two peptides derived from previously reported antigens (B13 & PFK).

### Results

Identification of Highly Conserved Trypanosoma cruzi Epitopes Towards Improved Diagnosis for Chagas Disease

### Peptide Selection

In total, 566 homologous sequence regions were extracted from T. cruzi genome alignments and 3,906 unique coding sequence annotations were identified within these homologous regions, corresponding to approximately 6 Mbp of genome sequence. 1,912 of these annotated sequences shared a nucleotide pairwise identity of !80% and were, thus, translated into protein sequences. After translation and removal of protein sequences with <90% pairwise identity across genomes, 1,756 highly conserved protein sequences remained. Finally, after removal of those sequences with a high pairwise identity (!90%) with another pathogen, 1,573 candidate antigens, corresponding to about 800,000 aas in total (10aa-4333aa, average length=496 aas) and around 10% of the proteome, were retained for further analysis. These candidate antigens had a mean pairwise identity of 97.1% (SD=0.022) across T. cruzi genomes and DTUS, and a low pairwise identity shared with other pathogens and humans (Table 5), except for T. rangeli. Unsurprisingly, as these two species are known to be very closely related, the mean identity shared between T. cruzi and T. rangeli across these sequences was somewhat higher.

TABLE 5
Mean Pairwise Identity of selected antigens across Genomes

| | T. | Leishmania | | T. | T. | T. | Homo |
|---|---|---|---|---|---|---|---|
| Pathogen | cruzi | spp. | P. vivax | gondii | brucei | rangeli | sapiens |
| Mean Pairwise | 97.1% | 40.3% | 8.7% | 11.8% | 46.7% | 67.9% | 9.9% |
| Identity (SD) | (0.02) | (18.9) | (15.6) | (19.9) | (19.8) | (13.9) | (17.7) |

More than half (799) of the candidate antigens selected for further analysis were hypothetical proteins. No single named protein group appeared to be over-represented. A few sequences identified in our analysis corresponded to previously reported CD antigens mined from the literature, namely JL8, hypothetical protein TcCLB.508385.10, and surface protein TolT, though this was excluded from further analysis because of high identity with other pathogen genomes. In large part, the sequences we identified were not known CD antigens. Of note, we did not identify known and commonly utilized Chagas antigens such as SAPA or TSSA through our analysis, likely because these did not meet the sequence conservation threshold that we established. We did, however, identify, three trans sialidase genes, known virulence factors that share immunodominant epitopes with SAPA. Our total list of candidate antigens also included ten putative calpain-like cysteine peptidases and eight flagellum associated proteins. Known T. cruzi antigen H49 is both a calpain-like protein and located in the flagellar attachment region, as are several other proteins also investigated as diagnostic antigens, suggesting that our peptide library contains good antigenic candidates.

### Microarray

Internal microarray positive controls performed comparably across all slides. While a few fields of the Cy3 controls showed large levels of background noise, there was a substantial difference between these averages and the Herpesvirus “SHRANETIYNTTLKY” positive control signals for each of the six slides (two slides per pool). HCMV peptides demonstrated natural variation in signal response of 15mers across the protein, as was also noted with our proteins and peptides. These results confirm that the microarray data is valid and comparable across the three sample pools.

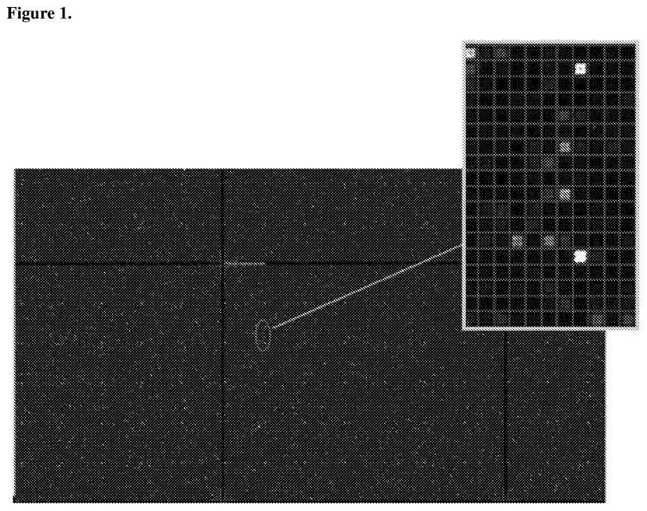

To analyze the antibody binding profile to T. cruzi antigens and peptides, we focused on the peptides for which antibody binding was the most different among our experimental groups, particularly the positive and negative control groups. Indeed, most peptides (>90%) in the high-density array presented no significant binding from any of the samples (FIG. 1), and only about 4,700 peptides showed differential recognition among our experimental groups (about 1% of peptides tested).

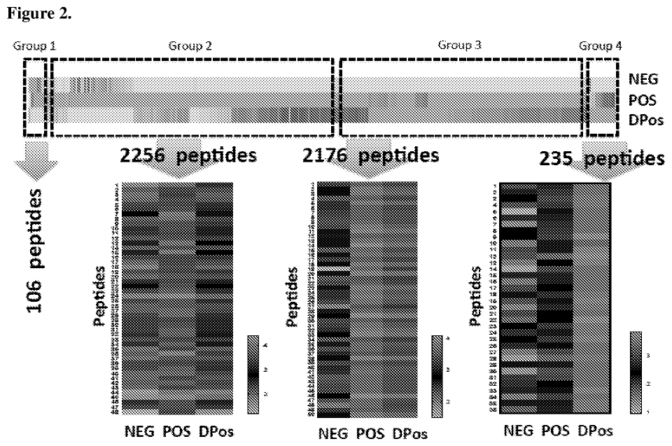

The high-density microarray demonstrated clear differences in the antibody binding profile of the different pools of samples tested against the selected conserved parasite antigens (FIG. 2). As expected, antibodies from the negative pool, representing healthy uninfected subjects, recognized only a few T. cruzi peptides (about 106 peptides). On the other hand, the positive pool from confirmed T. cruzi infected patients showed a high binding to a large number of the selected peptides (over 4,300 peptides). Finally, the pool of samples from positive patients that had been missed by current serological tests showed antibody binding to a more limited set of T. cruzi peptides. Importantly, comparing antibody profiles of the two positive sample pools indicated marked differences, clearly indicating that the immune response against these conserved peptides differed between these two groups of T. cruzi infected patients. Indeed, many peptides were recognized by positive control only (about 2256 peptides), and failed to show antibody binding from infected but discordant samples. Nonetheless, we could also identify a set of peptides against which all T. cruzi-infected pools presented high levels of antibodies. This set includes at least 2,176 peptides, which are good candidates for further development of an improved diagnostic test that will have increased sensitivity compared to current commercial tests (FIG. 2).

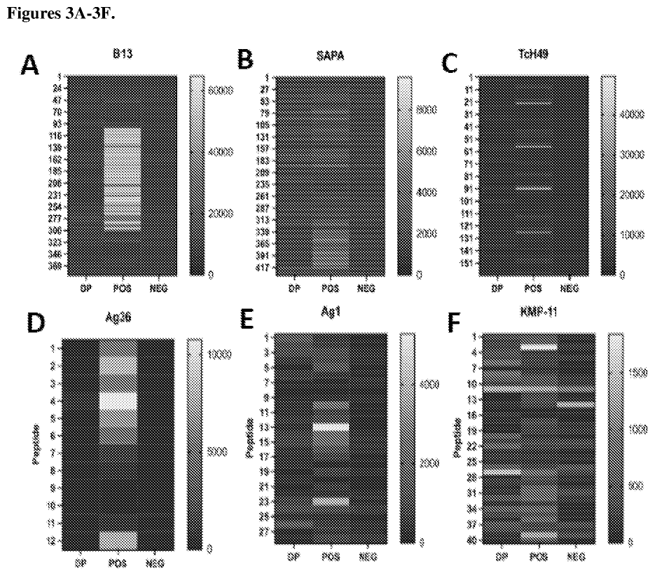

We further analyzed antibody binding profiles for specific antigens used in current commercial tests, such as antigens B13, SAPA, or PFK, to better understand their antigenicity (FIG. 3). We found strong and specific antibody binding to several of their derived peptides, mostly from the positive control sample pool, while the negative samples showed no, or insignificant binding as expected. Interestingly, samples from the infected but sero-discordant pool presented limited to no binding to these peptides and antigens, in agreement with the low sensitivity of commercial tests based on these antigens.

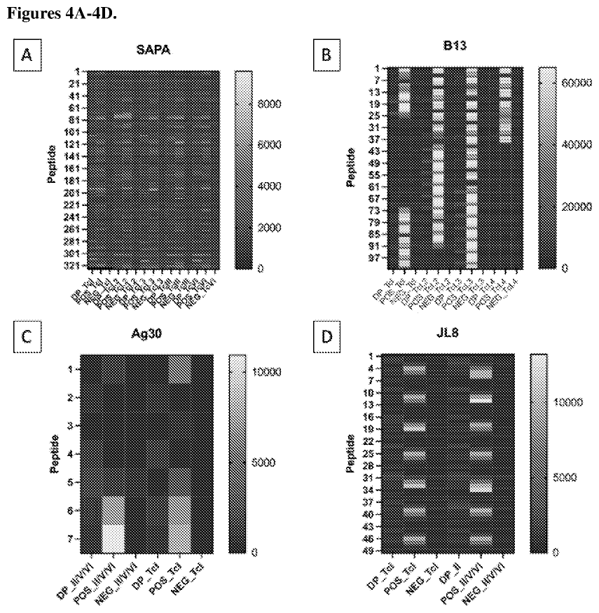

Because we had shown before that several of the antigens used in current commercial tests are not as conserved as initially thought, but present significant sequence variation between parasite strains and DTUs, we wanted to test if the differences in their antibody binding profiles between the positive control and the discordant but positive samples was due to this antigenic variation. Thus, we analyzed antibody binding profiles to sequence variants of antigens from several strains and DTUs. We included SAPA, B13, Antigen 30 and JL8 antigens (FIG. 4). As expected, the negative sample pool showed negligible antibody binding to these antigens. Also, for the positive sample pool, antibody binding to individual peptides among these antigens did somewhat vary according to parasite strains and DTUs, as expected due to their sequence variations, but the overall binding profile to each antigen was rather conserved and high for all sequence variants. Thus, antibodies from these positive controls are able to bind to these antigens irrespective of the strain or DTU they are derived from. Strikingly, antibodies from discordant but positive samples also failed to bind to all antigen sequence variants tested for B13, antigen 30 and JL8, and only showed limited binding to SAPA, but again this binding profile was very similar across strains and DTUs and always much lower compared to that obtained for the positive sample control. These results indicate that the differences in antibody recognition profile between positive controls and discordant but positive samples was not associated with antigenic variation. Furthermore, including sequence variants from multiple strains and DTUs of these antigens will not be sufficient to increase test sensitivity and adequately detect these discordant but positive samples, and new antigens need to be identified for this purpose.

### Selection of Candidate Peptides

As mentioned above, peptides that showed a high antibody binding from both the positive controls and the discordant but positive samples are good candidates for such an improved test. We selected 65 peptides for validation in an ELISA test (Table 6).

TABLE 6
Selected Candidate Peptides

| | | Sequence |
|---|---|---|
| Peptide ID | Peptide sequence | ID No. |
| P-TcCLB.506221.110 | MLSKNVDYFDGLRTFPRC | SEQ ID |
| | | NO: 1 |
| P-PFK | AKPPAESPFKSVFGAC | SEQ ID |
| | | NO: 2 |
| P-TcCLB.405165.10 | DAVKLEEFRFFIYERPSC | SEQ ID |
| | | NO: 3 |
| P-TcCLB.463297.10 | EESAASRPLYSFEAESLA | SEQ ID |
| | SHRC | NO: 4 |
| P-TcCLB.503419.54 | TEDGDFGWTVDVPIFLEC | SEQ ID |
| | | NO: 5 |
| P-TcCLB.503489.30 | KRCRDDTTPEYILSLGPQ | SEQ ID |
| | SAC | NO: 6 |
| P-TcCLB.503629.10 | SSTEIETFFLEGMSEC | SEQ ID |
| | | NO: 7 |
| P-TcCLB.503637.20 | AVSQLGEVENIKNKYSLH | SEQ ID |
| | IC | NO: 8 |
| P-TcCLB.506135.50 | RITNFILEGRHAHEKDGQ | SEQ ID |
| | GC | NO: 9 |
| P-TcCLB.506147.170 | TTYPPEDVYFPELRFC | SEQ ID |
| | | NO: 10 |
| P-TcCLB.506357.160 | LDAHQLEINRHLFESC | SEQ ID |
| | | NO: 11 |
| P-TcCLB.506401.170 | VADDGRVTPFYYDFEPLL | SEQ ID |
| | AC | NO: 12 |
| P-TcCLB.506513.24 | PPFKETQFSYFPSSDSC | SEQ ID |
| | | NO: 13 |
| P-TcCLB.506525.70 | ENDGYTQEHGIFQFGC | SEQ ID |
| | | NO: 14 |
| P-TcCLB.506533.34 | GDLIRRTLEEFSGDEC | SEQ ID |
| | | NO: 15 |
| P-TcCLB.506631.10 | KRIVSNMVPEIVDWVNC | SEQ ID |
| | | NO: 16 |
| P-TcCLB.506885.364 | GVVVFDLLRNEFTNEIFI | SEQ ID |
| | DEC | NO: 17 |
| P-TcCLB.506885.40 | CFDGGKRYWTYEDFDRFR | SEQ ID |
| | GYTC | NO: 18 |
| P-TcCLB.506937.40 | DKAYEWFPDSIQFLTC | SEQ ID |
| | | NO: 19 |
| P-TcCLB.506945.110 | NDVLTSRGETYDYQYSWC | SEQ ID |
| | | NO: 20 |
| P-TcCLB.507009.130 | RFRTAHEDSFYEFIFC | SEQ ID |
| | | NO: 21 |
| P-TcCLB.507093.90 | GYPEKPRDLFHEYLFC | SEQ ID |
| | | NO: 22 |
| P-TcCLB.507093.90.2 | RNMREESQTVDEFLFKRV | SEQ ID |
| | LLC | NO: 23 |
| P-TcCLB.507677.120 | SHTDLHFHAFQENYEDWC | SEQ ID |
| | | NO: 24 |
| P-TcCLB.508145.49 | AEAQHAMFDDDFLLFC | SEQ ID |
| | | NO: 25 |
| P-TcCLB.508277.380 | RNSASFLSGGEFNDWSNH | SEQ ID |
| | C | NO: 26 |
| P-TcCLB.508741.130 | VQDGDTKNTPGAFFFAEL | SEQ ID |
| | KGGC | NO: 27 |
| P-TcCLB.509555.10 | SALQPEHDELFFYRFC | SEQ ID |
| | | NO: 28 |
| P-TcCLB.510149.40 | DNFSGDATAGDRKVNC | SEQ ID |
| | | NO: 29 |
| P-TcCLB.510429.59 | DIATLRDGGYDFFIENC | SEQ ID |
| | | NO: 30 |
| P-TcCLB.510759.200 | AELLCGNAGNRKVNSLAC | SEQ ID |
| | | NO: 31 |
| P-TcCLB.510901.260 | DVAAKLQHPEFLFSDEDS | SEQ ID |
| | VLLC | NO: 32 |
| P-TcCLB.511115.20 | KLDVAFDFEVDAFLFC | SEQ ID |
| | | NO: 33 |
| P-TcCLB.511277.300 | MLREESPIIQMVWDAC | SEQ ID |
| | | NO: 34 |
| P-TcCLB.511277.420 | RTNESIFLENSEKWFC | SEQ ID |
| | | NO: 35 |
| P-TcCLB.511577.160 | NINAGREDILLFPLSRC | SEQ ID |
| | | NO: 36 |
| P-XP_820556.1 | PTSTDDEFQLIQLIQC | SEQ ID |
| | | NO: 37 |
| P-B13 | PAPFGQAAAGDKPSPC | SEQ ID |
| | | NO: 38 |
| P-B13new | DKPSPFGQAAAGDKPPPF | SEQ ID |
| | GC | NO: 39 |
| P-SAPAnew | SAHSTPSTPVDSSAHSTP | SEQ ID |
| | SC | NO: 40 |
| P-RiboP2 | AEEEEDDDMGFGLFDC | SEQ ID |
| | | NO: 41 |
| P-TSSA | GTDKKTAAGEAPSPSGC | SEQ ID |
| | | NO: 42 |
| P-RiboL19 | AKAAAAPAKAAAAPAC | SEQ ID |
| | | NO: 43 |
| P-P0 | EEEDDDDDFGMGALFC | SEQ ID |
| | | NO: 44 |
| P-R27-2b | ETEKQKAAEATKVAEC | SEQ ID |
| | | NO: 45 |
| P-Ag36 | QEDVGPRHVDPDHFRC | SEQ ID |
| | | NO: 46 |
| P-Ag1 | PEGVPLRELPLDDDSC | SEQ ID |
| | | NO: 47 |
| P-TcCLB.506773.120 | DFQETRPIPVWDLVYC | SEQ ID |
| | | NO: 48 |
| P-TcCLB.511303.150 | KGAPLYDDVFVPSFDC | SEQ ID |
| | | NO: 49 |
| P-TcCLB.504077.50 | VEPVCDDTQEYYFRLC | SEQ ID |
| | | NO: 50 |
| P-TcCLB.504109.100 | EIYLETIRNATNETIC | SEQ ID |
| | | NO: 51 |
| P-XP-815726.10 | TALRVSPYSIFLQELC | SEQ ID |
| | | NO: 52 |
| P-TcCLB.511671.50 | AAKPPAESPFRSVFGC | SEQ ID |
| | | NO: 53 |
| P-TcCLB.508831.150 | KPHAESPFKSVFGAPC | SEQ ID |
| | | NO: 54 |
| P-TcCLB.507009.10 | HSPRGSCKALLPEDAC | SEQ ID |
| | | NO: 55 |
| P-TcCLB.503805.6 | LRCVDSALEEAHLSIC | SEQ ID |
| | | NO: 56 |
| P-TcCLB.508175.110 | QFVPPWLQGYAFLPEC | SEQ ID |
| | | NO: 57 |
| P-XP_811353.1 | LRESDFVVNILPGTEC | SEQ ID |
| | | NO: 58 |
| P-TcCLB.506779.110 | EALTKDDTGNFWYSFC | SEQ ID |
| | | NO: 59 |
| P-TcCLB.507529.50 | VPKLFDLTYIPETSEC | SEQ ID |
| | | NO: 60 |
| P-TcCLB.508465.80 | AETAEPKEFFFDVDRC | SEQ ID |
| | | NO: 61 |
| P-TcCLB.506625.180 | GHLDDRVQIPDLFTLC | SEQ ID |
| | | NO: 62 |
| P-X57235 | GDARDWDILLAVGEVC | SEQ ID |
| | | NO: 63 |
| P-TcCLB.506127.150 | HKNDEGVTVSLVESLC | SEQ ID |
| | | NO: 64 |
| P-Q4D283 | VTREDEEYIVFSPQHC | SEQ ID |
| | | NO: 65 |

### Evaluation of Individual Peptide Performance Via Peptide ELISA

Several selected peptides failed to remain solubilized during the conjugation reaction and were discarded from use in the ELISA platform. For the remaining peptides, we evaluated diagnostic performance by testing individual plasma samples comprising the three pools used above for microarray screening. These included 11 confirmed negative samples, and 20 confirmed positive samples. This later group included 9 samples corresponding to positive controls (positive with current commercial tests), and 11 PCR positive samples but negative/missed by current commercial tests.

Antibody binding intensity was measured by the optical density at 450 nm (OD₄₅₀) and background values for wells incubated without samples were subtracted from all readings. The cut-off OD₄₅₀ value was established for each peptide as the mean OD₄₅₀ of the negative samples plus two standard deviations. We also performed Receiver Operating Characteristic (ROC) analyses for each peptide.

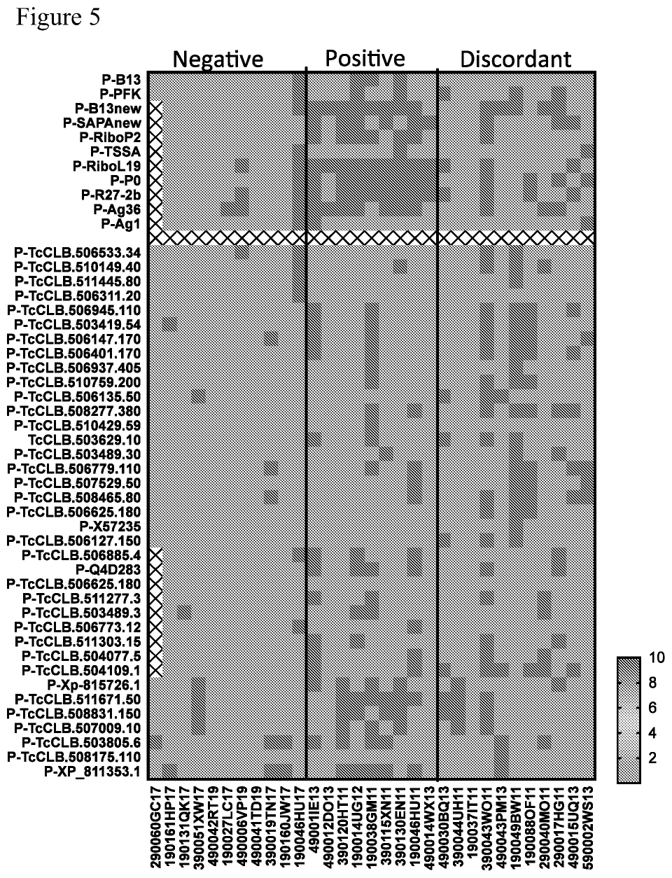

We first tested peptides derived from well-characterized antigens several of which are used in several current commercial tests (ELISA and rapid tests). These included B13, PFK, SAPA, TSSA, Riboprotein P2, Riboprotein L19, P0, R27-2, Antigen 36 and Antigen 1 (FIG. 5, top). We constructed heatmaps summarizing the reactivity (red) and non-reactivity (green) of samples against each peptide. Peptides derived from these antigens showed an overall good specificity as no or few negative samples reacted to these. Also, most positive control samples were reactive to these peptides as expected, although not all samples reacted to all peptides, indicating some individual variation among samples. More importantly, very few discordant samples reacted to these peptides, in agreement with the failure of current commercial tests to detect these discordant but positive samples.

We then evaluated additional peptides identified in our screening (FIG. 5, bottom). Most negative samples showed no or very limited binding to the peptides, indicating a high specificity. On the other hand, several positive control samples presented a high binding above the cut-off value, indicating peptide-specific antibodies in these samples. Nonetheless, some positive control samples also failed to react to some of the peptides. Multiple discordant samples also reacted with many of the peptides, indicating potential to increase diagnostic sensitivity. However, as mentioned above, we also detected extensive individual variation among plasma samples in their peptide recognition profile, as each sample had antibodies that recognized different peptides (FIG. 5). These results indicated that single peptide diagnostic is not feasible, but that a combination of peptides is needed for optimal diagnostic performance.

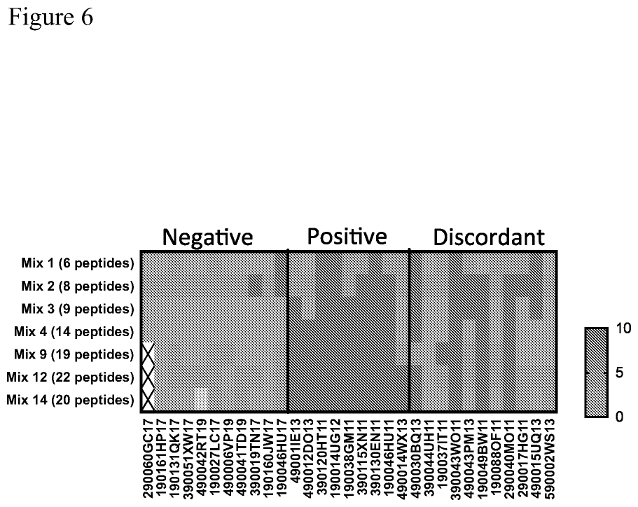

We then evaluated peptide combinations, and tested mixtures ranging from 6 to 22 peptides using the same set of plasma samples (FIG. 6). Performance was also compared to the FDA approved Wiener ELISA. As expected, we observed an overall increase in the reactivity of positive plasma samples to peptide mixtures as the number of peptides increased, while the reactivity of negative samples remained low, suggesting improved performance of peptide mixtures compared to single peptides. The optimum sensitivity and specificity was obtained with Mixtures 12 and 14, which included 22 and 20 peptides, respectively (Table 7). These reached a sensitivity of 6500 with a specificity of 10000. Compared to the performance of the commercial ELISA, this resulted in a 200% increase in sensitivity (4500 to 650%) while maintaining the high specificity (Table 7).

TABLE 7
Diagnostic performance of peptide mixtures and FDA approved ELISA.

| Mixture | Performance | 95% Confidence Interval |
|---|---|---|
| Mix 1 (6 peptides: SEQ ID | Sensitivity: 30.0% | 11.9% to 54.3% |
| NO: 2, 6, 10, 26, 51, 65) | Specificity: 90.9% | 58.7% to 99.8% |
| Mix 2 (8 peptides: SEQ ID | Sensitivity: 60.0% | 36.0% to 80.9% |
| NO: 2, 7, 9, 10, 26, 29, 34, | Specificity: 81.8% | 48.2% to 97.7% |
| 61) | | |
| Mix 3 (9 peptides: SEQ ID | Sensitivity: 65.0% | 40.8% to 84.6% |
| NO: 2, 7, 9, 10, 26, 29, 34, | Specificity: 100% | 71.5% to 100% |
| 54, 58) | | |
| Mix 4 (14 peptides: SEQ ID | Sensitivity: 60.0% | 36.0% to 80.9% |
| NO: 2, 7, 9, 10, 12, 20, 26, | Specificity: 100% | 71.5% to 100% |
| 29, 34, 38, 52, 53, 54, 58) | | |
| Mix 9 (19 peptides: SEQ ID | Sensitivity: 60.0% | 36.0% to 80.9% |
| NO: 6, 7, 9, 10, 12, 15, 20, | Specificity: 100% | 69.1% to 100% |
| 26, 29, 31, 34, 38, 52, 53, | | |
| 54, 55, 58, 62, 65) | | |
| Mix 12 (22 peptides: SEQ | Sensitivity: 65.0% | 40.8% to 84.6% |
| ID NO: 6, 7, 9, 10, 12, 15, | Specificity: 100% | 69.1% to 100% |
| 20, 26, 29, 31, 34, 39, 43, | | |
| 44, 45, 52, 53, 54, 55, 58, | | |
| 62, 65) | | |
| Mix 14 (20 peptides: SEQ | Sensitivity: 65.0% | 40.8% to 84.6% |
| ID NO: 6, 7, 9, 10, 12, 20, | Specificity: 100% | 69.1% to 100% |
| 26, 29, 31, 34, 39, 43, 45, | | |
| 52, 53, 54, 55, 58, 62, 65) | | |
| Wiener ELISA | Sensitivity: 45.0% | 23.0% to 68.5% |
| | Specificity: 100% | 69.1% to 100% |

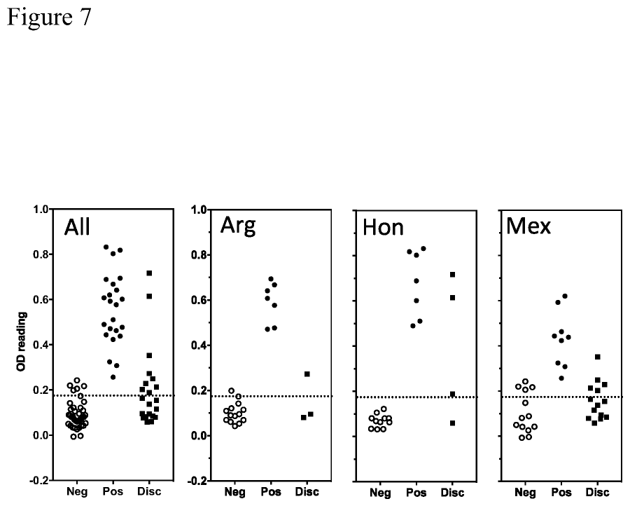

We then evaluated the performance of one of the best peptide mixture (Mixture 14) using a larger set of plasma samples (N=84) to better evaluate its performance, again comparing with a commercial ELISA test. These samples included 40 confirmed negative samples, and 44 confirmed positive samples. This later group included 23 samples corresponding to positive controls (positive with current commercial tests), and 21 PCR positive samples but negative/missed by current commercial tests (discordant samples). As expected, negative control samples presented very low/no reactivity against the peptide mixture, while positive controls presented a high reactivity (FIG. 7). In addition, many discordant samples were reactive to the peptides with a high intensity, indicating a clear improvement in the sensitivity of the assay. Indeed, the peptide ELISA assay reached a sensitivity of 72.7% with a specificity of 87.5% (Table 8).

Importantly, this improvement was observed in samples from all countries tested, namely Argentina, Honduras and Mexico (FIG. 7). Indeed, the proportion of discordant samples identified as reactive was ⅓ (33.3%) for samples from Argentina, ¾ (75%) for samples from Honduras, and 5/14 (35.7%) for samples from Mexico. Compared with the diagnostic performance of a FDA-approved commercial ELISA, the peptide mixture presented a clear improvement in sensitivity, while its specificity remained high (Table 8). Adding additional peptides from those identified in the microarray can further increase test performance.

TABLE 8
Diagnostic performance of peptide ELISA with extended sample set.

| Mixture 14 | | Wiener ELISA | |
|---|---|---|---|
| Parameter | 95% CI | Parameter | 95% CI |
| Sensitivity: 72.7% | 57.2% to 85.0% | Sensitivity: 52.3% | 36.7% to 67.5% |
| Specificity: 87.5% | 73.2% to 95.8% | Specificity: 100% | 91.2% to 100% |
| PPV: 86.1% | 72.8% to 93.5% | PPV: 52.4% | 41.2% to 72.2% |
| NPV: 72.9% | 62.7% to 81.2% | NPV: 100% | |
| Accuracy: 78.6% | 68.3% to 86.8% | Accuracy: 75% | 64.3% to 83.8% |

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting. The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context. The terms “comprise”, “have”, “include” and “contain” (and their variants) are open-ended linking verbs and allow the addition of other elements when used in a claim.

The use of the word “a” or “an” when used in conjunction with the term “comprising” in the claims or the specification means one or more than one, unless the context dictates otherwise.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method. The use of the term “or” in the claims is used to mean “and/or” unless explicitly indicated to refer to alternatives only or if the alternatives are mutually exclusive.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

1. A composition comprising a peptide or a peptide mixture having antigenic properties with respect to Trypanosoma cruzi (T. cruzi), the composition comprising:
at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65; or
at least one analog, homolog, or antigenic fragment of one or more of SEQ ID NO:1-SEQ ID NO:65; or
a peptide or peptide mixture comprising at least one peptide having at least 70% sequence identity to one or more of SEQ ID NO:1-SEQ ID NO:65; or
a combination thereof.

2. The composition of claim 1, comprising a peptide mixture consisting of:
SEQ ID NO:2, 6, 10, 26, 51, and 55; or
at least one analog, homolog, or antigenic fragment of one or more of the group of sequences consisting of SEQ ID NO:2, 6, 10, 26, 51, and 55; or
a peptide or peptide mixture comprising at least one peptide having at least 70% sequence identity to one or more of the group of sequences consisting of SEQ ID NO:2, 6, 10, 26, 51, and 55; or
a combination thereof.

3. The composition of claim 1, comprising a peptide mixture consisting of:
SEQ ID NO:2, 7, 9, 10, 26, 29, 34, and 61; or
at least one analog, homolog, or antigenic fragment of one or more of the group of sequences consisting of SEQ ID NO:2, 7, 9, 10, 26, 29, 34, and 61; or
a peptide or peptide mixture comprising at least one peptide having at least 70% sequence identity to one or more of the group of sequences consisting of SEQ ID NO:2, 7, 9, 10, 26, 29, 34, and 61; or
a combination thereof.

4. The composition of claim 1, comprising a peptide mixture consisting of:
SEQ ID NO:SEQ ID NO:2, 7, 9, 10, 26, 29, 34, 54, and 58; or
at least one analog, homolog, or antigenic fragment of one or more of the group of sequences consisting of SEQ ID NO:2, 7, 9, 10, 26, 29, 34, 54, and 58; or
a peptide or peptide mixture comprising at least one peptide having at least 70% sequence identity to one or more of the group of sequences consisting of SEQ ID NO:2, 7, 9, 10, 26, 29, 34, 54, and 58; or
a combination thereof.

5. The composition of claim 1, comprising a peptide mixture consisting of:
SEQ ID NO:2, 7, 9, 10, 12, 20, 26, 29, 34, 38, 52, 53, 54, and 58; or
at least one analog, homolog, or antigenic fragment of one or more of the group of sequences consisting of SEQ ID NO:2, 7, 9, 10, 12, 20, 26, 29, 34, 38, 52, 53, 54, and 58; or
a peptide or peptide mixture comprising at least one peptide having at least 70% sequence identity to one or more of the group of sequences consisting of SEQ ID NO:2, 7, 9, 10, 12, 20, 26, 29, 34, 38, 52, 53, 54, and 58; or
a combination thereof.

6. The composition of claim 1, comprising a peptide mixture consisting of:
SEQ ID NO:6, 7, 9, 10, 12, 15, 20, 26, 29, 31, 34, 38, 52, 53, 54, 55, 58, 62, and 65; or
at least one analog, homolog, or antigenic fragment of one or more of the group of sequences consisting of SEQ ID NO:6, 7, 9, 10, 12, 15, 20, 26, 29, 31, 34, 38, 52, 53, 54, 55, 58, 62, and 65; or
a peptide or peptide mixture comprising at least one peptide having at least 70% sequence identity to one or more of the group of sequences consisting of SEQ ID NO:6, 7, 9, 10, 12, 15, 20, 26, 29, 31, 34, 38, 52, 53, 54, 55, 58, 62, and 65; or
a combination thereof.

7. The composition of claim 1, comprising:
a peptide mixture consisting of SEQ ID NO:6, 7, 9, 10, 12, 15, 20, 26, 29, 31, 34, 39, 43, 44, 45, 52, 53, 54, 55, 58, 62, and 65; or
at least one analog, homolog, or antigenic fragment of one or more of the group of sequences consisting of SEQ ID NO:6, 7, 9, 10, 12, 15, 20, 26, 29, 31, 34, 39, 43, 44, 45, 52, 53, 54, 55, 58, 62, and 65; or
a peptide or peptide mixture comprising at least one peptide having at least 70% sequence identity to one or more of the group of sequences consisting of SEQ ID NO:6, 7, 9, 10, 12, 15, 20, 26, 29, 31, 34, 39, 43, 44, 45, 52, 53, 54, 55, 58, 62, and 65; or
a combination thereof.

8. The composition of claim 1, comprising:
a peptide mixture consisting of SEQ ID NO:6, 7, 9, 10, 12, 20, 26, 29, 31, 34, 39, 43, 45, 52, 53, 54, 55, 58, 62, and 65; or
at least one analog, homolog, or antigenic fragment of one or more of the group of sequences consisting of SEQ ID NO:6, 7, 9, 10, 12, 20, 26, 29, 31, 34, 39, 43, 45, 52, 53, 54, 55, 58, 62, and 65; or
a peptide or peptide mixture comprising at least one peptide having at least 70% sequence identity to one or more of the group of sequences consisting of SEQ ID NO:6, 7, 9, 10, 12, 20, 26, 29, 31, 34, 39, 43, 45, 52, 53, 54, 55, 58, 62, and 65; or
a combination thereof.

9. A method for detecting a T. cruzi antibody in a biological sample, comprising:
obtaining a biological sample from a subject;
contacting the biological sample with the composition according to claim 1; and
detecting and/or quantifying the T. cruzi antibody bound to the composition.

10. The method of claim 9, including selecting the biological sample from the group consisting of tissue, blood, serum, saliva and urine.

11. The method of claim 9, including detecting and/or quantifying the T. cruzi antibody by a suitable immunoassay.

12. A method for diagnosing Chagas disease and/or a T. cruzi infection, comprising:
obtaining a biological sample from a subject;
contacting the biological sample with the composition according to any one of claim 1;
detecting and/or quantifying one or more T. cruzi antibody bound to the composition; and
according to the quantifying, performing additional confirmatory diagnostic analyses.

13. The method of claim 12, including selecting the biological sample from the group consisting of tissue, blood, serum, saliva and urine

14. The method of claim 12, including detecting and/or quantifying the T. cruzi antibody by a suitable immunoassay.

15. The method of claim 12, including selecting the subject from a group of subjects having or at risk of having one of an acute phase of Chagas disease, a chronic phase of Chagas disease, an indeterminate phase of Chagas disease, or a congenital T. cruzi infection.

16. The method of claim 12, wherein the subject has previously tested serologically negative for T. cruzi antibodies.

17. An apparatus for determination and/or quantification of one or more T. cruzi antibodies, comprising:
a suitable substrate; and
the composition according to claim 1, wherein the peptide or peptide mixture is immobilized on the suitable substrate to provide a microarray.

18. The apparatus of claim 17, wherein the peptide or peptide mixture is conjugated to a suitable carrier protein for the immobilization.

19. (canceled)

20. A kit for determination and/or quantification of one or more T. cruzi antibodies, comprising:
the apparatus of claim 17;
optionally, packaging for the apparatus;
optionally, one or more suitable preservatives for the peptide or peptide mixture; and
optionally, instructional materials for the use of the apparatus.

21. A method for identifying T. cruzi contamination, comprising:
obtaining a biological sample;
contacting the biological sample with the composition according to claim 1; and
detecting one or more T. cruzi antibodies bound to the composition.

22-23. (canceled)
